(19) United States
(12) Patent Application Publication
(10) Pub. No.: US 2024/0151680 A1
(43) Pub. Date: May 9, 2024

(54) ELECTROCHEMICAL SENSOR DEVICE FOR RAPID ANALYTE DETECTION AND METHODS OF MAKING AND USING THE SAME

(71) Applicant: Nevada Research & Innovation Corporation, Reno, NV (US)

(72) Inventors: Manoranjan Misra, Reno, NV (US); Bhaskar Vadlamani, Reno, NV (US)

(73) Assignee: Nevada Research & Innovation Corporation, Reno, NV (US)

(21) Appl. No.: 18/279,562

(22) PCT Filed: Feb. 24, 2022
(86) PCT No.: PCT/US2022/017721

§ 371 Date: Aug. 30, 2023

Related U.S. Application Data
(60) Provisional application No. 63/155,080, filed on Mar. 1, 2021.

(51) Int. Cl. G01N 27/327

(52) U.S. Cl. CPC G01N 27/3278

(57) ABSTRACT

Disclosed herein are embodiments of a electrochemical sensor device for rapidly determining whether a sample comprises analytes of interest. In particular embodiments, the analytes of interest are biomarkers associated with a physiological condition or disease. The electrochemical sensor device comprises a substrate-based platform having a working electrode comprising functionalized nanotubes that are functionalized with a metal ion and/or a polymer component. In some embodiments, the functionalized nanotubes comprise nanotubes prepared by a double anodization method that provides nanotubes having an average length greater than 3 μm. In some additional embodiments, the substrate-based platform can comprise a substrate made of a fiber-based material.

### CROSS REFERENCE TO RELATED APPLICATIONS

This application claims the benefit of the earlier priority date of U.S. Provisional Application No. 63/155,080, filed Mar. 1, 2021, which is incorporated by reference herein in its entirety.

### FIELD

The present disclosure is directed to electrochemical sensor device embodiments for rapid analyte (e.g., biomarkers and other compounds of interest) detection and methods of making and using such sensor device embodiments.

### BACKGROUND

Sensor devices for detecting biomarkers associated with particular diseases, such as diabetes and tuberculosis, have been developed in the field of diagnostics; however, currently available devices suffer from drawbacks that detract from their use in non-invasive diagnostic methods, such as poor detection limits and/or unreliable responses. Such devices also require assembly methods or complex components/configurations that result in high manufacturing costs and the inability to produce single-use devices. There exists a need in the art of diagnostics for new devices that exhibit improved detection limits, selectivity and specificity, and that can be made in a cost-effective manner, particularly for point-of-use care.

### SUMMARY

Disclosed herein are embodiments of a substrate-based platform, comprising: a substrate comprising a fiber-based material or a solid support material that is not fiber-based; a working electrode comprising a plurality of functionalized nanotubes, wherein the functionalized nanotubes comprise metal oxide-based nanotubes functionalized with a metal ion species and/or an electroactive polymer component; a reference electrode; and a counter electrode.

Also disclosed herein are embodiments of a sensor device, comprising: a substrate-based platform as described herein; and a potentiostat.

Also disclosed are embodiments of a method, comprising: applying a voltage to a sensor device as described herein; exposing the sensor device to a sample; and sensing and/or measuring a change in current produced by the sensor device after being exposed to the sample.

Also disclosed are embodiments of a method of making a working electrode as described herein, the method comprising: performing a first anodization of a metal substrate to obtain the metal oxide-based nanotubes; performing a second anodization of the metal oxide-based nanotubes to increase the length of the metal oxide-based nanotubes; and depositing the metal ion species and/or the electroactive polymer component on the metal oxide-based nanotubes to provide the plurality of functionalized nanotubes.

The foregoing and other objects and features of the present disclosure will become more apparent from the following detailed description, which proceeds with reference to the accompanying figures.

### BRIEF DESCRIPTION OF THE DRAWINGS

FIG. 1 is an illustration of a representative substrate-based platform according to the present disclosure.

FIG. 2 is a schematic illustration showing how sensor device embodiments disclosed herein can be used for analyte detection, including biomarker detection.

FIG. 3 is a schematic illustration showing how sensor device embodiments disclosed herein can be used in combination with a breath bag for analyte (e.g., biomarker) detection in condensed breath samples.

FIG. 4 is a micrograph obtained from scanning electron microscopic (SEM) analysis of a nanotube array comprising TiO₂ nanotubes made using a double anodization, wherein the micrograph shows a cross-section of the TiO₂ nanotubes that were milled using a focused-ion beam (FIB) technique, and wherein the nanotubes have an average length of 3 μm.

FIG. 5 is an SEM micrograph showing the top view of a functionalized nanotube array wherein TiO₂ nanotubes were functionalized with a polyaniline polymer.

FIG. 6 is a graph obtained using energy-dispersive x-ray spectroscopy (EDS) which provides an elemental analysis of the functionalized nanotubes of FIG. 5.

FIG. 7 is an SEM micrograph showing the top view of a functionalized nanotube array wherein TiO₂ nanotubes were functionalized with a polyaniline polymer and cobalt ions.

FIG. 8 is a graph obtained using energy-dispersive x-ray spectroscopy (EDS) which provides an elemental analysis of the functionalized nanotubes of FIG. 7.

FIG. 9 is a graph of current (mA) as a function of time (seconds) and that shows the amperometry response obtained using a substrate-based platform comprising a plurality of functionalized nanotubes comprising TiO₂ nanotubes functionalized with cobalt ions to detect a methyl nicotinate biomarker at a bias voltage of −0.5 V.

FIG. 10 is a graph of current (mA) as a function of time (seconds) and that shows the amperometry response obtained using a substrate-based platform comprising a plurality of functionalized nanotubes comprising TiO₂ nanotubes functionalized with a polyaniline polymer to detect a methyl nicotinate biomarker at a bias voltage of −0.5 V.

FIG. 11 is a graph of current (mA) as a function of time (seconds) and that shows the amperometry response obtained using a substrate-based platform comprising a plurality of functionalized nanotubes comprising TiO₂ nanotubes functionalized with a polyaniline polymer and cobalt ions to detect a methyl nicotinate biomarker at a bias voltage of −0.5 V.

FIG. 12 is a bar graph showing the responses obtained when substrate-based platforms comprising (i) a plurality of cobalt ion-functionalized TiO₂ nanotubes (“Co-TiNT”), (ii) a plurality of polyaniline polymer-functionalized TiO₂ nanotubes (“PANI-TiNT”), and (iii) a plurality of functionalized TiO₂ nanotubes comprising polyaniline and cobalt ions (“Co-PANI-TiNT”) are used to evaluate a sample comprising 0.1 mM of methyl nicotinate.

### DETAILED DESCRIPTION

### I. Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” and “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and compounds similar or equivalent to those described herein can be used in the practice or testing of the present disclosure, suitable methods and compounds are described below. The compounds, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Ratios, concentrations, amounts, and other numerical data may be expressed herein in a range format. It is to be understood that such a range format is used for convenience and brevity, and thus, should be interpreted in a flexible manner to include not only the numerical values explicitly recited as endpoints of the range, but also to include all the individual numerical values or sub-ranges encompassed within that range. Furthermore, not all alternatives recited herein are equivalents.

To facilitate review of the various embodiments of the disclosure, the following explanations of specific terms are provided.
Analyte: A substance of interest that is detected, measured, and/or identified using an electrochemical sensor device according to the present disclosure. In particular disclosed embodiments, analytes according to the present disclosure are biomarkers, including (but not limited to) biomarkers for infectious diseases, like tuberculosis.
Biomarker: A measurable indicator of a physiological disease or condition. In particular embodiments, a biomarker can also be a volatile organic compound (or “VOC”).
Cellulosic Fiber Material: A material comprising cellulose or a derivative thereof that can be obtained from natural materials or sources.
Functionalized Nanotubes: A plurality of nanotubes functionalized with a metal ion species and/or an electroactive polymer. In some embodiments, the plurality of nanotubes can be functionalized with the metal ion species and/or the electroactive polymer such that a coating of the metal ion species and/or the electroactive polymer is positioned along a top surface of the plurality. In some embodiments, individual nanotubes of the plurality can be functionalized with the metal ion species and/or the electroactive polymer such that the surface (including external and/or interior surfaces) of one or more of the individual nanotube comprises a coating of the metal ion species and/or the electroactive polymer.
Nanotube: A nanometer-scale tube-like structure that typically is hollow.
Synthetic Fiber Material: A material that is not found in nature, but mimics physical and/or chemical properties of a natural plant or animal fiber.
Substrate-Based Platform: A component of an electrochemical sensor device of the present disclosure that comprises a substrate that is made of a fiber-based material or a substrate made of a solid material that is not fiber-based and that further comprises one or more electrodes.
Volatile Organic Compound: A compound that typically is emitted as a gas from a solid or a liquid. In some embodiments, a volatile organic compound can comprise a biomarker, but need not always comprise a biomarker.

### II. Device Embodiments

Disclosed herein is an electrochemical sensor device for use in sensing and/or identifying analytes in a sample. In particular embodiments, the electrochemical sensor device is designed to detect biomarkers associated with infectious diseases, such as tuberculosis. The disclosed electrochemical sensor device can be used as a single-use device or as a multi-use device. In some embodiments, the electrochemical sensor device embodiments disclosed herein can be made with low-cost materials and can be fabricated efficiently and thus avoid the complex fabrication methods and high manufacturing costs associated with current electrochemical sensor devices, particularly those that have been developed for tuberculosis biomarker detection. Device embodiments disclosed herein are fast, user friendly, inexpensive, and, in some embodiments, disposable. Device embodiments constructed for single-use can be made using cost-effective paper-based materials, which can provide a thin, mechanically stabilized film of a sample that facilitates delivering analytes to electrode surfaces by capillary action. And, the continuous wicking of the sample and any analytes therein across the electrodes of the sensor device helps avoid the mass transport limitations of conventional devices and provides improved detection limits using electrochemical analytical techniques, such as cyclic voltammetry and amperometry.

In particular embodiments, the sensor device comprises a substrate-based platform. The substrate-based platform can comprise a substrate made of a fiber-based material or a substrate made of a solid material that is not fiber-based. In some embodiments, the fiber-based material can comprise a cellulosic fiber material capable of wicking, such as a paper-type material, or a synthetic fiber material. In some such embodiments, the cellulosic fiber material can be obtained from any source, such as from natural materials (e.g., wood, hemp, linen, cotton, or the like). In some embodiments, the cellulosic fiber material can be a cellulose paper or other porous paper material. In some embodiments, the synthetic fiber materials can include, but are not limited to, a polyester fiber material, an acrylic fiber material, or the like. In yet other embodiments, the substrate-based platform can further comprise a film material. In some such embodiments, the film material can comprise a thermally conductive polyimide film (e.g., a Kapton® film sold by DuPont), or other transparent plastic and/or polymeric materials. In some embodiments, the film material can be used as a support material for other components of the substrate-based platform, or it can be used to adhere such components to the substrate component of the substrate-based platform. In some other embodiments, the substrate-based platform can further comprise one or more additional substrates for physical support, such as a substrate made of a plastic material (e.g., biaxially oriented polypropylene (BOPP) or high-density polyethylene (HDPE)), a metal material (e.g., non-conductive metal materials and/or conductive metal materials); or a glass material. In independent embodiments, the substrate-based platform can comprise such solid materials for use as a solid support upon which other components of the platform can be positioned (e.g., the electrodes discussed below). In such embodiments, the substrate-based platform does not need to comprise a fiber-based material.

In particular embodiments, the substrate-based platform further comprises a plurality of electrodes attached to, or printed onto, the substrate. The electrodes can include a working electrode, a counter electrode, and a reference electrode. The reference electrode can be any suitable reference electrode, such as an Ag/AgCl reference electrode. The counter electrode can be any suitable counter electrode, such as a titanium electrode. The working electrode comprises a plurality of functionalized nanotubes. The functionalized nanotubes comprise nanotubes that have been functionalized with a metal ion component, a polymer component, or a combination thereof. In particular embodiments, the functionalized nanotubes are functionalized with the polymer component or both the polymer component and the metal ion component. In representative embodiments, the functionalized nanotubes are functionalized with the polymer component and the metal ion component. Functionalization of the nanotubes can occur either by coating a surface of the plurality of nanotubes with the metal ion species and/or the polymer component or by coating a surface of one or more individual nanotubes of the plurality. Coating can involve physical contact and/or chemical contact through ionic and/or non-ionic bonds. The nanotubes that become functionalized are synthesized to have an average length ranging from 1 μm to 4 μm, such as 2 μm to 4 μm, or 3 μm to 4 μm. In particular embodiments, the nanotubes have an average length of greater than 3 μm. In particular embodiments, nanotubes having these lengths provide an advantage over shorter nanotubes having lengths below 3 μm as the longer nanotubes exhibit increased surface area and thus can promote more chemical reactions between analytes (e.g., biomarkers) and the components that functionalize the nanotubes (e.g., the metal ions and/or the polymer component).

The nanotubes are made of one or more metal oxides. The metal oxide can be a transition metal oxide or a main-group metal oxide. Transition metal oxides can include, but are not limited to, a titanium oxide, a tantalum oxide, an iron oxide, a zinc oxide, a copper oxide, a nickel oxide, a chromium oxide, a vanadium oxide, a manganese oxide, a zirconium oxide, a palladium oxide, a platinum oxide, a cobalt oxide, a silver oxide, a magnesium oxide, or combinations thereof. Main-group metal oxides can include, but are not limited to an aluminum oxide, a silicon oxide, a gallium oxide, an indium oxide, a tin oxide, a lead oxide, or combinations thereof. In representative embodiments, the nanotubes comprise a titanium oxide, such as titanium dioxide (as referred to herein as titania or TiO₂); a tantalum oxide, such as TaO₂, Ta₂O₅, or a combination thereof; a tin oxide (e.g., SnO₂); a zinc oxide (e.g., ZnO); or a combination thereof.

In particular embodiments, the nanotubes are functionalized with a polymer component. The polymer component typically is an electroactive polymer; however, other materials can be used, including conductive polymer materials, conductive membrane materials, and/or conductive adhesive materials. In some embodiments, the polymer component is an electroactive polyaniline polymer or a salt thereof. In some embodiments, the polymer component can be an electroactive polyaniline polymer having an average M_{w} ranging from 1,000 to 100,000, such as 5,000 to 75,000, or 5,000 to 65,000, or 5,000 to 50,000, or 5,000 to 20,000. In particular embodiments, the electroactive polymer is a polyaniline polymer, such as emeraldine base polyaniline, emeraldine salt polyaniline, leucoemeraldine base polyaniline, or the like.

In some embodiments, the nanotubes are further functionalized with at least one metal ion species. The metal ion species is one that is capable of binding an analyte present in a sample, such as a biomarker compound associated with an infectious disease. For example, in some embodiments, the metal ion species can be selected to bind a tuberculosis biomarker compound, such as methyl nicotinate, methyl phenylacetate, methyl p-anisate, and o-phenylanisole. In some embodiments, the metal ion species can be selected from cobalt ions, copper ions, lithium ions, iron ions, nickel ions, lead ions, chromium ions, manganese ions, scandium ions, antimony ions, titanium ions, arsenic ions, platinum ions, gold ions, zinc ions, palladium ions, silver ions, or combinations thereof and including any mono-, di-, tri-, or tetravalent ion species thereof. In some embodiments, the metal ion species can be selected from Ag¹⁺, Au¹⁺, Cu¹⁺, Fe¹⁺, Li¹⁺, Co²⁺, Cu²⁺, Fe³⁺, Pd²⁺, Pb²⁺, Zn²⁺, Fe³⁺, Co³⁺, Cr³⁺, Mn³⁺, Ni³⁺, Sb³⁺, Sc³⁺, As⁴⁺, Mn⁴⁺, Ni⁴⁺, Pd⁴⁺, Pt⁴⁺, Sb⁴⁺, Ti⁴⁺ or combinations thereof. In particular embodiments, the metal ion species includes cobalt ions, such as Co²⁺ or Co³⁺, or a combination thereof; gold ions, such as Au¹⁺; silver ions, such as Ag¹⁺; copper ions, such as Cu¹⁺, Cu²⁺, or combinations thereof; or nickel ions, such as Ni³⁺, Ni⁴⁺, or combinations thereof. In some embodiments, the metal ion species can be selected based on the ability of such ions to bind with a target biomarker compound, which can be determined using computational modeling and/or by using cyclic voltammetry. For example, in some embodiments, the nanotubes can be functionalized with cobalt ions to promote detecting tuberculosis biomarkers, such as methyl nicotinate, methyl phenylacetate, methyl p-anisate, and o-phenylanisole.

In particular embodiments, the nanotubes are coated with the polymer component and the metal ion species to provide the functionalized nanotubes. In exemplary embodiments, the nanotubes are coated first with the polymer component and then with the metal ion species to provide the functionalized nanotubes. In some embodiments, the metal ion species can be provided by electrodeposition or by metal ion exchange. Such methods are described herein. In an independent embodiment, the substrate-based platform can comprise unfunctionalized nanotubes that are free of any metal ion species and/or any polymer component. In yet other independent embodiments, the substrate-based platform does not comprise a metal ion species without further comprising a polymer component.

The sensor device can further comprise other components in addition to the substrate-based platform, such as a power source, a potentiostat, a housing, a sample introduction inlet or region, connective components (e.g., wires, clamps, adhesives, or the like), a regeneration source, control mechanisms, and/or electronic displays. In particular embodiments, the sensor device comprises a power source that is integrated with the sensor device or that is separate from the sensor device. For example, in embodiments comprising an integrated power source, it can be a built-in rechargeable, disposable, or replaceable battery. In embodiments using an external power source, it can be connected to the sensor device through wires or can be paired with the sensor device using other means. The power source can be a direct current or alternating current power source. In particular embodiments, the power source is a battery and in some embodiments can be a battery of a cellular device or other portable electronic device. The power source is configured to supply sufficient power so as to generate a voltage (such as a bias voltage) suitable for actuating the sensor device. In some embodiments, the power generated from the power source can be tuned to provide a particular voltage that is selected depending on the type of analyte that the device is being used to detect. In some embodiments, a voltage suitable for biomarkers of infectious diseases, such as tuberculosis can include a voltage ranging from −0.1 V to −1.2 V, such as −0.2 V to −1.2 V, or −0.3 V to −1 V, or −0.5 V to −1 V, or −0.6 V to −1V, or −0.3 V to −0.5 V. In some embodiments, the voltage can range from −0.1 V to −0.7 V, such as −0.45 V to −0.35 V, or −0.5 V to −0.7 V. In particular embodiments, the sensor device further comprises a potentiostat that is integrated with the sensor device and that can facilitate measuring any current (or change in current) generated by the sensor device during use.

In embodiments where the sensor device is configured to be reusable, the sensor device can also include a regeneration source configured to regenerate components of the working electrode (e.g., the functionalized nanotubes). In some embodiments, the regeneration source can be an ultraviolet light source that can be activated to direct ultraviolet light on the functionalized nanotubes after using the sensor device. In such embodiments, the ultraviolet light can cause the analyte compounds bound to the metal ions of the functionalized nanotubes to be released so that the sensor device can be reused. In some other embodiments, a transparent housing can be used in combination with an external ultraviolet light source to facilitate regeneration.

The sensor device also can include integrated controls or it can be configured to be controlled by a personal computer, laptop, smart phone, or other smart electronic devices. The sensor device can further comprise an electronic display that can be used to view results from the sensor device. In some embodiments, the sensor device can display a graphical representation of the current signal from the sensor device, or it can provide a verbal cue to indicate whether or not an analyte (e.g., a biomarker) is present (e.g., “present” or “not present”; “positive” or “negative”; “yes” or “no”; or the like); an audio cue (e.g., a beep or other alarm indicating that a biomarker is present); or any combination thereof. In some embodiments, the sensor device can be encased within a housing that may contain all components or only certain components of the sensor device. In particular embodiments, the housing may contain the substrate-based platform, the potentiostat, and the power source, if the power source is integrated. In embodiments comprising a housing, an opening is provided so as to facilitate delivery of the sample to the substrate and/or the working electrode comprising the plurality of nanotubes. Also, as described above, the housing can be transparent so as to facilitate regeneration of the working electrode.

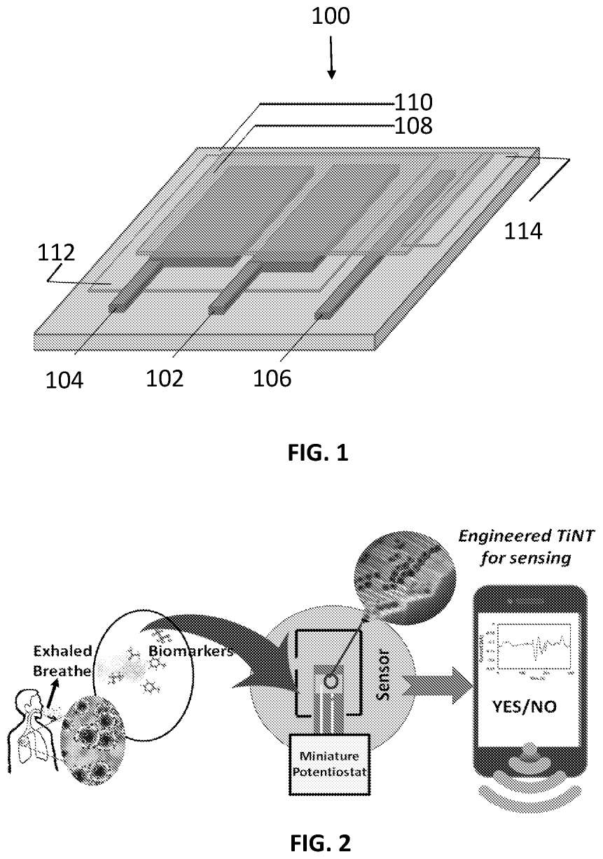

Components and configurations used in an exemplary device embodiment are illustrated schematically in FIG. 1. FIG. 1 provides an illustration of a substrate-based platform component of a device embodiment. As shown in FIG. 1, substrate-based platform 100 can comprise working electrode 102, counter electrode 104, and reference electrode 106. Working electrode 102 comprises a plurality of functionalized nanotubes (not illustrated). Substrate-based platform 100 further comprises cellulosic fiber material 108 and glass substrate 110, which can serve as a cover and/or physical support, along with a film material 112. Film material 112 (e.g., a double-sided conductive tape) can be used to adhere working electrode 102 and counter electrode 104 to glass substrate 110 and adhesive material 114 can be used to couple the glass substrate and cellulosic fiber material 108.

### III. Method Embodiments

Disclosed herein are embodiments of a method of making and using the electrochemical sensor device embodiments disclosed herein. In particular, method embodiments for making the functionalized nanotubes are described, along with methods of using the sensor device.

The functionalized nanotubes of the sensor device can be made using an anodization method in combination with one or more deposition methods (e.g., electrodeposition). In some embodiments, the nanotubes themselves are made using a double anodization method. In such embodiments, two separate anodization steps are used in combination with one or more annealing steps to provide nanotubes comprising the metal oxide material. The nanotubes made with this double anodization method exhibit increased lengths relative to nanotubes made with a single anodization step. In particular embodiments, a first anodization step is performed, followed by an optional washing step, and then heating the sample at a temperature ranging from 80° C. to 120° C., such as 100° C., for at least one hour (e.g., 1, 2, 3, or 4 hours). In some embodiments, a first annealing step in an oxygen atmosphere at 500° C. also can be used after the first anodization step. This process is then repeated for a second time, with the second anodization step being performed at sub-zero temperatures and being followed by an annealing step in the presence of oxygen at a temperature ranging from 200° C. to 600° C., such as 300° C. to 575° C., or 400° C. to 550° C., or 450° C. to 500° C. for 1 to 10 hours, such as 1, 2, 3, 4, 5, 6, 7, 8, 9, or 10 hours. In some embodiments, the process can be repeated two, three, four, or five times.

In some embodiments, the method can further comprise depositing a metal ion species on the nanotubes by exposing the nanotubes to a metal ion precursor solution after a second anodization step and before a second annealing step. After treating the nanotubes with the metal ion precursor solution, they can be exposed to heat for a suitable time (e.g., 80° C. to 120° C., such as 100° C., for 60 minutes to 4 hours, such as 1, 2, or 3 hours). The nanotubes can then be exposed to the second annealing step.

The nanotubes also can be functionalized with a polymer component. One or more deposition methods can be used to functionalize the nanotubes with the polymer component. In particular embodiments, a plurality of nanotubes is prepared as described above, and can be prepared with or without the metal ion species. The nanotubes are then exposed to a polymer-containing solution and allowed to mix for a time period. After an optional rinsing step with water, an electrodeposition step is performed by applying a voltage to the composition for a suitable time period. In some embodiments, electrodeposition is performed by exposing the solution comprising the nanotubes and the polymer to a constant potential ranging from −0.5 V to −1.4 V, such as −0.8 V to −1.2 V, or −0.9 V to −1.1 V for a time period ranging from 10 minutes to 30 minutes (e.g., 5 minute to 15 minutes). The resulting functionalized nanotubes can be washed and dried.

In some embodiments, the nanotubes can be exposed to multiple deposition steps to provide functionalized nanotubes comprising a deposited metal ion species and polymer component. In such embodiments, the method can comprise performing a double anodization method as described above and then performing separate electrodeposition steps to provide the deposited polymer component and the metal ion species. In particular embodiments, after double anodization, the nanotubes are exposed to a polymer solution and electrodeposition is carried out as described above. Then, metal ion species can be deposited by exposing the polymer-functionalized nanotubes to a metal ion precursor solution and then performing electrodeposition at a constant potential ranging from −0.8 V to −1.2 V, such as −0.85 V to −0.95 V, or −0.9 V to −1.1 V.

In particular embodiments, the sensor device can be constructed to be compatible in methods for detecting or identifying the presence of a particular analyte, including volatile and/or non-volatile biomarker compound and/or volatile organic compounds that are not (or do not comprise) biomarkers. For example, a device embodiment can be constructed to comprise substrate-based platform having a working electrode that is covered with a plurality of functionalized nanotube comprising a particular metal ion species that binds a particular analyte. In particular embodiments, the sensor device is constructed for use in detecting analytes present in fluid samples, particularly liquid samples. The sample can be a biological sample obtained from a subject or can be a non-biological sample. In particular embodiments, the biological sample can be condensed breath, saliva, mucous, or other forms of biological samples that can be obtained and provided in liquid form. In representative embodiments, the sample is condensed breath, a saliva sample, or other biological sample obtained from a subject. In embodiments where a condensed breath sample is used, the sample can be collected by having a subject breath into a breath bag or other breath-capturing device and then obtaining condensate therefrom. As such, rather than testing a subject's breath using gas analysis of the breath with the sensor device, liquid-based analysis can be used. In particular disclose embodiments, the cellulosic-fiber based or synthetic fiber material forming the substrate of the substrate-based platform can be used to wick the condensed breath along a path of the substrate-based platform so that it contacts the working electrode. Similarly, saliva from a subject can be analyzed using the sensor device. In some embodiments, the sample can be dissolved or diluted with water prior to analysis using the sensor device.

In particular embodiments, the sensor device can be used to detect the presence of biomarkers associated with physiological conditions or diseases and thus can be used to diagnose a subject that has, or that may develop, the condition or disease. In some embodiments, the disease or condition can be, but is not limited to, tuberculosis, breast cancer, lung cancer, heart disease, diabetes, preeclampsia, oxidative stress, transplant rejection, ischemic heart disease, or combinations thereof. In particular embodiments, the sensor device is used to sense the presence of analytes that might be present in a sample that are biomarkers for tuberculosis, such as methyl nicotinate, methyl phenylacetate, methyl p-anisate, o-phenylanisole, or combinations thereof. The sensor device also can be used to detect other analytes that can be biomarkers for other diseases and/or conditions, including (but not limited to), acetone, alkane compounds, alkene compounds, ammonia, mercaptans, fatty acids, lactic acid, uric acid, urease, reduced or oxidized forms of glutathione, and the like.

Particular method embodiments of using the sensor device to detect, identify, and/or quantify an analyte present in a sample is disclosed. In some embodiments, the method comprises applying a voltage to the sensor device, measuring a current produced by the sensor device, exposing the sensor device to a sample, sensing a change in current produced by the sensor device, and measuring the change in current. In some embodiments, the method can further comprise condensing the sample prior to exposing the sensor device to the sample. In yet additional embodiments, the method can further comprise diagnosing a subject having, or is at risk of developing, a physiological condition or disease using results produced by the sensor device. In yet some additional embodiments, the method can comprise exposing the sensor device to ultraviolet light to facilitate release of analyte compounds that may bind to the functionalized nanotubes. This step can be used for device embodiments that are intended for multiple uses.

A voltage can be applied to the sensor device using a power source of the sensor device, which may be a separate component or integrated with the sensor device. The applied voltage can be as described herein, with particular embodiments having an applied voltage ranging from −0.1 V to −0.7 V, such as −0.45 V to −0.35 V, or −0.5 V to −0.7 V. Current produced by the sensor device can be measured using any suitable means for measuring an electrical current. In some embodiments, cyclic voltammetry is used. In yet other embodiments, amperometry is used. In some embodiments, cyclic voltammetry and amperometry can be used. In particular embodiments, the current is measured to obtain a baseline current that is emitted by the sensor device with or without being exposed to a sample. The current also may be measured to determine the presence of, the identity of, or the amount of, an analyte present in a sample. In particular embodiments, the current produced by the sensor device is measured using the potentiostat and displayed on a computer or other display device electrically connected to the potentiostat.

The sensor device can be exposed to the sample by physically associating the sample with the sensor device in any suitable manner so as to facilitate chemical, electrical, and/or physical contact between the sample and the functionalized nanotubes of the working electrode. The sensor device can comprise a sample introduction region and/or a sample introduction inlet that is used to apply the sample to the sensor device for use. In some embodiments, the sensor device is configured in a manner such that the sample can be introduced into the sensor device so as to be in direct contact with the working electrode. In yet other embodiments, the sensor device is configured in a manner such that the sample comes into contact with the working electrode through wicking or capillary action provided by the cellulosic fiber material or the synthetic fiber material of the substrate-based platform. In particular embodiments, the sensor device is exposed to the sample using a swab to administer a liquid sample to the sensor device. In such embodiments, the swab can be used to directly swab a sample from the subject by swabbing the subject's mouth or nose and then contacting the sensor device with the swab. In other embodiments, the swab can be used to swab a liquid sample that has been obtained from a subject and subsequently stored in a container (e.g., a breath bag or other breath-capturing device). In yet additional embodiments, the sensor device can be exposed to the sample by administering a liquid sample obtained from a breath bag or other breath-capturing device onto the sensor device. The amount of sample needed for use with the device is minimal and in some embodiments, the amount can be less than 1000 nanograms/liter, such as 1 nanogram/liter to 1000 nanograms/liter, or 4 nanograms/liter to 1000 nanograms/liter, or 4 nanograms/liter to 100 nanograms/liter, or 1 nanogram/liter to 50 nanograms/liter.

After the sensor device has been exposed to a sample, a change in current produced by the sensor device can be sensed and/or measured. In particular embodiments, the change in current can comprise an increase of current from the current observed or measured prior to sample addition. In such embodiments, the term “increase” means that the current becomes more negative. In some embodiments, the change in current can be measured. In such embodiments, measuring the change in current produced by the sensor device after exposing it to the sample can indicate that a particular analyte is present in the sample. The sensor device can be used for selective analyte detection as it can provide a signal upon binding of the desired analyte to the functionalized nanotubes, whereas other compounds that might be present in the sample do not bind and thus do not provide any signal. In some embodiments, measuring the change in current produced by the sensor device after exposing it to the sample can facilitate identifying the analyte in terms of its chemical identity. For example, a particular analyte may provide a particular current change and thus the value of the current change can be used to characterize the analyte. In yet additional embodiments, measuring the change in current produced by the sensor device after exposing it to the sample can provide an indication as to how much of the analyte is present in the sample.

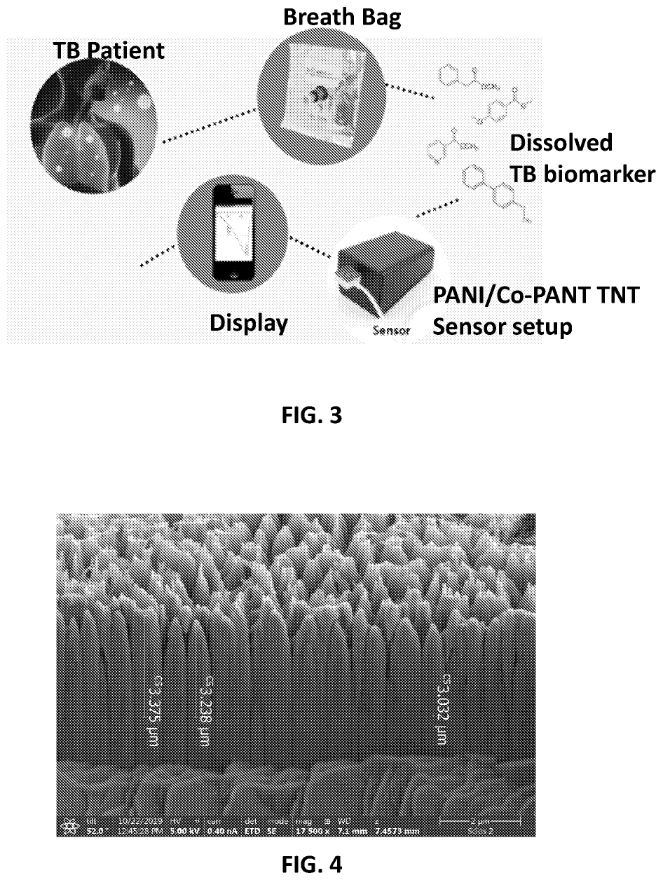

FIGS. 2 and 3 are schematics showing how device embodiments disclosed herein can be used in the above described method embodiments. As illustrated schematically in FIG. 2, condensed breath from a subject can be collected and added to a sensor device comprising a working electrode with functionalized nanotubes, which can bind analytes present in the condensed breath and the sensor device will sense a current change upon binding of the analyte of interest. A result signal (e.g., “yes” or “no” message) is then conveyed to a user via a display. FIG. 3 illustrates another embodiment wherein a breath bag is used to collect breath condensate from a subject which is then analyzed after being exposed to a sensor device in a housing. The results are then displayed for the user.

In some embodiments, cyclic voltammetry can be used to verify the binding of an analyte to the functionalized nanotubes. In some embodiments, particular analytes of interest can be biomarkers that comprise particular functional groups that can be detected electrochemically using cyclic voltammetry. In some embodiments, the biomarkers comprise a functional group that can be oxidized using an appropriate electrolyte system (e.g., a supporting salt, such as perchlorate) with pH adjustment. After oxidation, the biomarker can yield distinct anodic waves with peaks occurring at different potentials. From the integrated anodic current vs. time, the charge released can be calculated. Concentrations of the biomarkers can be determined from the anodic charge calculations using Faraday's law with an assumption that all the charges are attributed to oxidation of the biomarkers.

### IV. Overview of Several Embodiments

Disclosed herein are embodiments of a substrate-based platform, comprising: a substrate comprising a fiber-based material or a solid support material that is not fiber-based; a working electrode comprising a plurality of functionalized nanotubes, wherein the functionalized nanotubes comprise metal oxide-based nanotubes functionalized with a metal ion species and/or an electroactive polymer component; a reference electrode; and a counter electrode.

In some embodiments, the fiber-based material is a cellulosic fiber-based material or a synthetic fiber-based material.

In any or all of the above embodiments, the fiber-based material is paper obtained from wood, hemp, linen, cotton, or combinations thereof.

In any or all of the above embodiments, the substrate comprises the solid support material and wherein the solid support material is selected from a plastic material, a glass material, or a metal material.

In any or all of the above embodiments, the metal oxide-based nanotubes of the plurality of functionalized nanotubes have an average length greater than 3 μm.

In any or all of the above embodiments, the metal oxide-based nanotubes comprise a titanium oxide, a tantalum oxide, an iron oxide, a zinc oxide, a copper oxide, a nickel oxide, a chromium oxide, a vanadium oxide, a manganese oxide, a zirconium oxide, a palladium oxide, a platinum oxide, a cobalt oxide, a silver oxide, a magnesium oxide, or combinations thereof.

In any or all of the above embodiments, the metal oxide-based nanotubes comprises TiO₂, SnO₂, TaO₂, Ta₂O₅, or ZnO.

In any or all of the above embodiments, the metal ion species is selected from a cobalt ion, a copper ion, a lithium ion, an iron ion, a nickel ion, a lead ion, a chromium ion, a manganese ion, a scandium ion, an antimony ion, a titanium ion, an arsenic ion, a platinum ion, a gold ion, a zinc ion, a palladium ion, a silver ion, or combinations thereof and including any mono-, di-, tri-, or tetravalent ion species thereof.

In any or all of the above embodiments, the metal ion species is Co²⁺, Co³⁺, Au¹⁺, Ag¹⁺, Cu¹⁺, Cu²⁺, Ni³⁺, Ni⁴⁺, or a combination thereof.

In any or all of the above embodiments, the electroactive polymer component is a polyaniline polymer or a salt thereof.

In any or all of the above embodiments, the polyaniline polymer has an average Mw ranging from 1,000 to 100,000.

In any or all of the above embodiments, the reference electrode is an Ag/AgCl electrode and the counter electrode is a titanium electrode.

In any or all of the above embodiments, the substrate-based platform further comprises a potentiostat.

In any or all of the above embodiments, the substrate comprises a cellulosic fiber material; the working electrode comprises a plurality of functionalized nanotubes, wherein the functionalized nanotubes comprise TiO₂-based nanotubes functionalized an electroactive polyaniline polymer; the reference electrode is Ag/AgCl; and the counter electrode is titanium.

In any or all of the above embodiments, the functionalized nanotubes are further functionalized with cobalt ions.

Also disclosed herein are embodiments of a sensor device, comprising: the substrate-based platform according to any or all of the above substrate-based platform embodiments; and a potentiostat.

In any or all of the above embodiments, the sensor device further comprises a power source.

In any or all of the above embodiments, the sensor device further comprises a sample introduction inlet or region; a housing; or a combination thereof.

Also disclosed herein are embodiments of a method, comprising: applying a voltage to a sensor device according to any or all of the above sensor device embodiments; exposing the sensor device to a sample; and sensing a change in current produced by the sensor device after being exposed to the sample.

In any or all of the above method embodiments, the voltage is applied to the sensor device using a power source.

In any or all of the above method embodiments, the power source is integrated in the sensor device or wherein the power source is an external power source.

In any or all of the above method embodiments, exposing the sensor device to the sample comprises contacting the working electrode of the sensor device with the sample.

In any or all of the above method embodiments, the substrate-based platform of the sensor device comprises the substrate comprising the fiber-based material and wherein the sample is a liquid and contacting the working electrode comprises placing the sample on the substrate-based platform of the sensor device such that the liquid flows over the working electrode by wicking or capillary action.

In any or all of the above method embodiments, the sample is a biological sample and the method further comprises collecting the biological sample from a subject.

In any or all of the above method embodiments, the biological sample is condensed breath, saliva, or other biological material from a subject.

In any or all of the above method embodiments, the method further comprises measuring the change in current produced by the sensor device.

In any or all of the above method embodiments, the change in current produced by the sensor device signifies a binding event between an analyte present in the sample and the functionalized nanotubes of the working electrode, wherein the analyte is a volatile organic compound.

In any or all of the above method embodiments, the volatile organic compound is, or comprises, a biomarker selected from methyl nicotinate, methyl phenylacetate, methyl p-anisate, o-phenylanisole, or combinations thereof.

In any or all of the above method embodiments, the method further comprises diagnosing a subject from which the biological sample is obtained, wherein the subject has, or is at risk of developing, a physiological condition or disease.

In any or all of the above method embodiments, the disease is tuberculosis.

Also disclosed are embodiments of a method for making a working electrode as described herein for any or all of the above embodiments, the method comprising: performing a first anodization of a metal substrate to obtain the metal oxide-based nanotubes; performing a second anodization of the metal oxide-based nanotubes to increase the length of the metal oxide-based nanotubes; and depositing the metal ion species and/or the electroactive polymer component on the metal oxide-based nanotubes to provide the plurality of functionalized nanotubes.

### V. EXAMPLES

### Example 1

In this example, nanotubes comprising TiO₂ were prepared. The nanotubes were prepared by anodizing Ti foils (1 square inch) in an electrolytic solution comprising 0.5 wt % NH₄F and 5 vol % H₂O in ethylene glycol under an ultrasonically agitated condition using an ultrasonic bath (100 W, 42 KHZ, Branson 2510R-MT). A two-electrode configuration was used for anodization. A flag shaped platinum (Pt) electrode served as a cathode. The anodization was carried out by applying a potential of 20 V using a rectifier (Agilent, E3640A) for 30 minutes. The as-anodized TiO₂ templates were washed with water and then placed in an oven at 100° C. for 3 hours. The anodization process was then repeated, followed by annealing in oxygen at 500° C. for 2 hours. In particular examples, a sub-zero temperature was maintained during anodization to avoid artifacts on the titanium substrate. In particular examples, the nanotubes were formed into an ordered array of vertically-oriented and free standing TiO₂ oxide nanotubes. FIG. 4 provides an SEM micrograph showing a cross-section of a nanotube array made according to this example, wherein the average length of the nanotubes was observed to be at least 3 μm.

### Example 2

In this example, TiO₂ nanotubes functionalized with cobalt ions were made. Anodization was performed on a titanium sheet as described above, followed by baking in an oven at 100° C. for 3 hours. The second anodization was conducted on the same sample as described above. Then, 0.48 M of CoCl₂·6H₂O solution was smeared onto the sample, followed by baking at 100° C. for 3 hours. Subsequently, the sample was annealed at 500° C. for 3 hours in oxygen atmosphere.

### Example 3

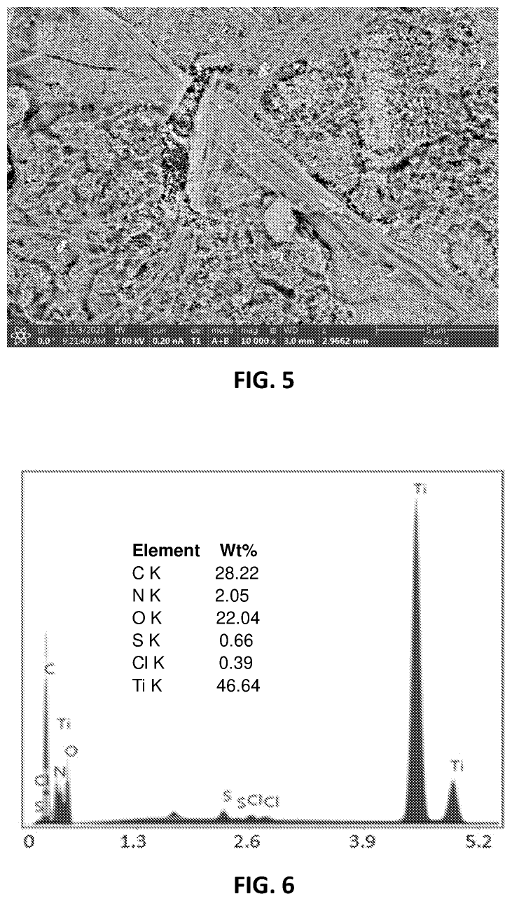

In this example, TiO₂ nanotubes functionalized with a polyaniline polymer were made. The TiO₂ nanotubes were prepared according to the anodization procedure of Example 1. Then the TiO₂ nanotubes were dipped into an acetone-based solution comprising 0.4 μM polyaniline and 0.5 M H₂SO₄. The solution was stirred at speed of 200 rpm for 20 hours. In some other embodiments, the nanotubes are immersed in the acetone-based solution for less than 20 hours (e.g., 1 to 5 hours) and then the process is repeated a second time. The sample was then rinsed in DI water. Electrodeposition was carried out in the same solution at a constant potential of −1 V for 10 minutes. The sample was then washed with DI water followed by ethanol and dried under air flow. The SEM micrograph and EDS map of the sample are shown in FIGS. 5 and 6, respectively.

### Example 4

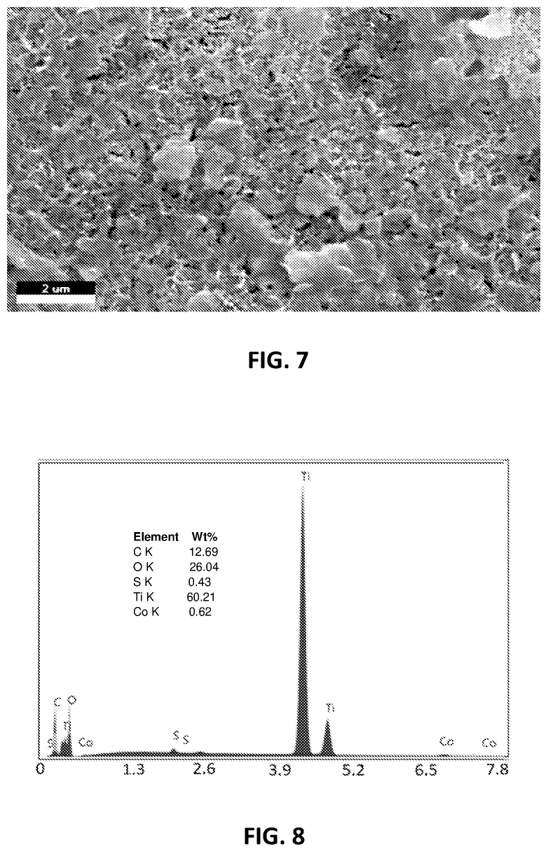

In this example, cobalt was deposited onto functionalized TiO₂ nanotubes comprising an electroactive polyaniline polymer using a potentiostatic electrodeposition technique. The functionalized nanotubes comprising the polyaniline polymer were prepared according to Example 3. After electrodeposition of the polymer and prior to any washing step, the functionalized nanotubes were exposed to a cobalt solution and cobalt ions were electrodeposited using a potential of −0.9 V (vs Ag/AgCl). The SEM micrograph and EDS map of the sample are shown in FIGS. 7 and 8, respectively.

### Example 5

In this example, a device embodiment is constructed. First, an Ag/AgCl conductive ink is painted onto a glass substrate and baked at 100° C. for 15 minutes to form the Ag/AgCl reference electrode. Then, a double-sided Kapton® tape is placed onto the glass substrate, next to the Ag/AgCl reference electrode. A plurality of functionalized nanotubes is added onto the Kapton® tape next to the Ag/AgCl electrode to form the working electrode. A titanium sheet also is added onto the Kapton® tape next to the working electrode. A cellulose paper is placed on top of the three electrodes and glued to the glass substrate with a single-sided Kapton® tape. For testing, an electrical contact to the functionalized TiO₂ nanotubes is made using alligator clips. Aluminum foil is placed on Ag/AgCl electrode at its edge and electrical contact is made by clamping alligator clip onto the aluminum.

### Example 6

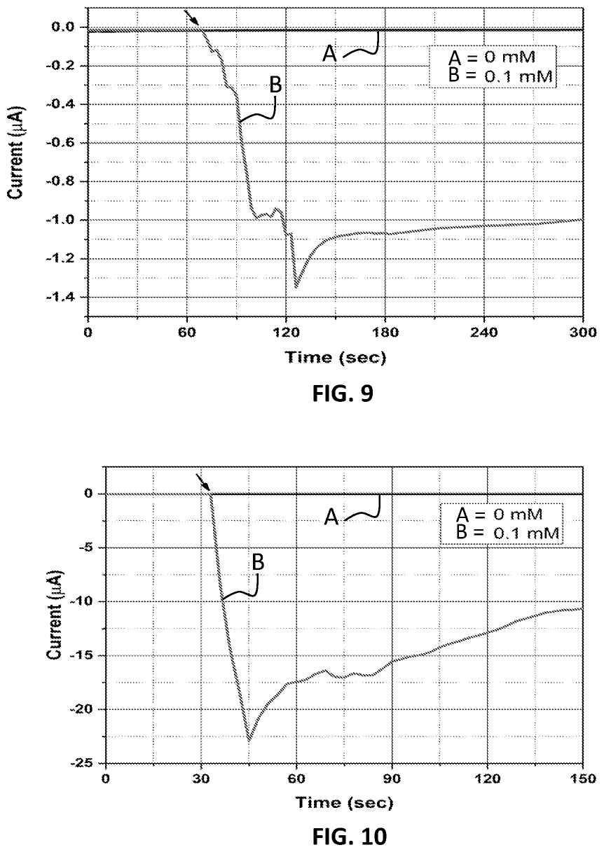

In this example, the ability of Co-functionalized TiO₂ nanotubes to detect methyl nicotinate, which is a biomarker for tuberculosis, was determined by conducting amperometry testing at a bias voltage of −0.5 V. Results are shown in FIG. 9. Upon addition of 0.1 mM of methyl nicotinate (marked by an arrow in FIG. 9), the substrate-based platform response current immediately increased to −1.34 μA. The baseline current corresponding to 0 mM (background) of methyl nicotinate is found to be ˜10 pA.

### Example 7

In this example, the ability of polyaniline-functionalized TiO₂ nanotubes to detect methyl nicotinate was evaluated. The electrochemical response of a substrate-based platform comprising polyaniline-coated TiO₂ nanotubes is shown in FIG. 10. With methyl nicotinate addition (marked by an arrow in FIG. 10), the substrate-based platform response current increased to −23 μA. The baseline current corresponding to no addition of methyl nicotinate was observed to be 10 pA. The response current with the polyaniline-functionalized TiO₂ nanotubes was 17 times higher than that with the embodiment of Example 6.

### Example 8

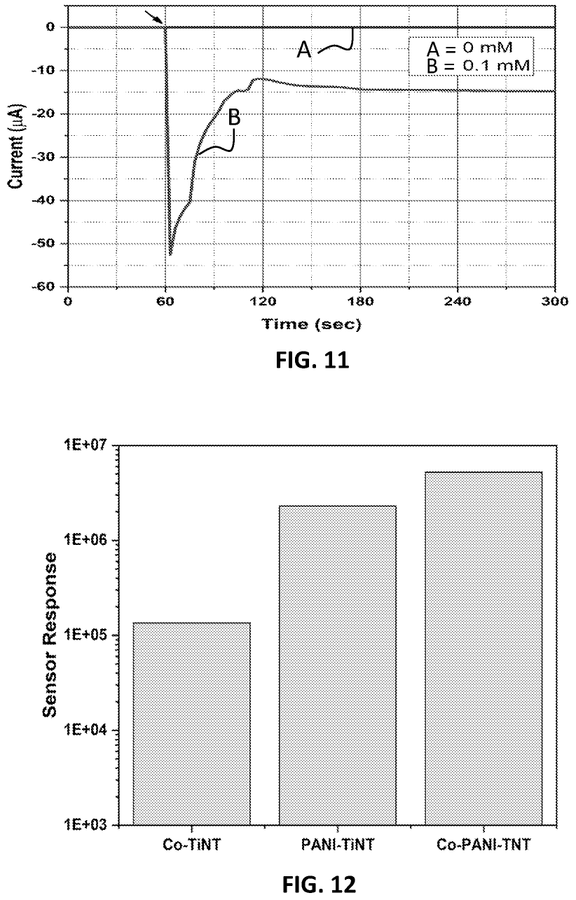

In this example, the ability of a substrate-based platform comprising TiO₂ nanotubes functionalized with a polyaniline polymer and cobalt ions to detect the presence of methyl nicotinate was evaluated. An amperometry electrochemical technique was used, with results shown in FIG. 11. The addition of the methyl nicotinate tuberculosis biomarker at 60 seconds (marked by an arrow in FIG. 11), resulted in rapid increase in the substrate-based platform response current to 52 μA. The observed current was much high compared to that of Examples 6 and 7. The increase in current response is 40 times higher than Example 6 and 2.3 times higher than Example 7, indicating that in particular embodiments, the combination of polyaniline and cobalt is very sensitive to the biomarkers.

The response for substate-based platforms made according to this example and for Examples 6 and 7 were calculated using the following equation:

Response (R)=(i_{max,biomarker}−i_{max,baseline})÷i_{max,baseline} where i_{max,biomarker} is the maximum response current obtained when the substrate-based platform is exposed to the tuberculosis biomarker and i_{max,baseline} is the maximum response current obtained when the substrate-based platform is not exposed to the tuberculosis biomarker. The value of i_{max,baseline}, which is the current noted when the substrate-based platform is not exposed to the tuberculosis biomarker, is found to be ˜10 pA. The response obtained for the substrate-based platform embodiments of Examples 6-8 are summarized in FIG. 12. In particular, the response obtained when 0.1 mM of methyl nicotinate was exposed to the substrate-based platform of Example 6 (labeled as “Co-TiNT” in FIG. 12), and Example 7 (labeled as “PANI-TiNT” in FIG. 12), and Example 8 (labeled as “PANI-Co-TiNT” in FIG. 12) was determined to be 1.35×10⁵, 2.3×10⁶ and 5.2×10⁶, respectively.

In view of the many possible embodiments to which the principles of the present disclosure may be applied, it should be recognized that the illustrated embodiments are only preferred examples and should not be taken as limiting the scope of the present disclosure. Rather, the scope is defined by the following claims. We therefore claim as our invention all that comes within the scope and spirit of these claims.

1. A substrate-based platform, comprising:
a substrate comprising a fiber-based material or a solid support material that is not fiber-based;
a working electrode comprising a plurality of functionalized nanotubes, wherein the functionalized nanotubes comprise metal oxide-based nanotubes functionalized with a metal ion species and/or an electroactive polymer component;
a reference electrode; and
a counter electrode.

2. The substrate-based platform of claim 1, wherein the fiber-based material is a cellulosic fiber-based material or a synthetic fiber-based material.

3. The substrate-based platform of claim 1, wherein the fiber-based material is paper obtained from wood, hemp, linen, cotton, or combinations thereof.

4. The substrate-based platform of claim 1, wherein the substrate comprises the solid support material and wherein the solid support material is selected from a plastic material, a glass material, or a metal material.

5. The substrate-based platform of claim 1, wherein the metal oxide-based nanotubes of the plurality of functionalized nanotubes have an average length greater than 3 μm and comprise a titanium oxide, a tantalum oxide, an iron oxide, a zinc oxide, a copper oxide, a nickel oxide, a chromium oxide, a vanadium oxide, a manganese oxide, a zirconium oxide, a palladium oxide, a platinum oxide, a cobalt oxide, a silver oxide, a magnesium oxide, or combinations thereof.

6. The substrate-based platform of claim 1, wherein the metal oxide-based nanotubes comprises TiO₂, SnO₂, TaO₂, Ta₂O₅, or ZnO.

7. The substrate-based platform of claim 1, wherein the metal ion species is selected from a cobalt ion, a copper ion, a lithium ion, an iron ion, a nickel ion, a lead ion, a chromium ion, a manganese ion, a scandium ion, an antimony ion, a titanium on, an arsenic ion, a platinum ion, a gold ion, a zinc ion, a palladium ion, a silver ion, or combinations thereof and including any mono-, di-, tri-, or tetravalent ion species thereof.

8. The substrate-based platform of claim 7, wherein the metal ion species is Co²⁺, Co³⁺, Au¹⁺, Ag¹⁺, Cu¹⁺, Cu²⁺, Ni³⁺, Ni⁴⁺, or a combination thereof.

9. The substrate-based platform of claim 1, wherein the electroactive polymer component s a polyaniline polymer or a salt thereof.

10. The substrate-based platform of claim 9, wherein the polyaniline polymer has an average M_{w} ranging from 1,000 to 100,000.

11. The substrate-based platform of claim 1, wherein the reference electrode is an Ag/AgCl electrode and the counter electrode is a titanium electrode, and wherein the substrate-based platform further comprises a potentiostat.

12. The substrate-based platform of claim 1, wherein:
the substrate comprises a cellulosic fiber material;
the working electrode comprises a plurality of functionalized nanotubes, wherein the functionalized nanotubes comprise TiO₂-based nanotubes functionalized an electroactive polyaniline polymer;
the reference; electrode is Ag/AgCl; and
the counter electrode is titanium.

13. The substrate-based platform of claim 12, wherein the functionalized nanotubes are further functionalized with cobalt ions.

14. A sensor device, comprising:
the substrate-based platform of claim 1; and
a potentiostat.

15. The sensor device of claim 14, further comprising a sample introduction inlet or region; a housing; a power source, or a combination thereof.

16. A method, comprising:
applying a voltage to a sensor device according to claim 14
exposing the sensor device to a sample; and
sensing a change in current produced by the sensor device after being exposed to the sample.

17. The method of claim 16, wherein the voltage is applied to the sensor device using a power source, wherein the power source is integrated in the sensor device or wherein the power source is an external power source.

18. The method of claim 16, wherein exposing the sensor device to the sample comprises contacting the working electrode of the sensor device with the sample, wherein the change in current produced by the sensor device is sensed and measured.

19. The method of claim 18, wherein the substrate-based platform of the sensor device comprises the substrate comprising the fiber-based material and wherein the sample is a liquid and contacting the working electrode comprises placing the sample on the substrate-based platform of the sensor device such that the liquid flows over the working electrode by wicking or capillary action.

20. The method of claim 16, wherein the sample is a biological sample selected from condensed breath, saliva, or other biological material and the method further comprises collecting the biological sample from a subject.

21. (canceled)

22. The method of claim 18, wherein the change in current produced by the sensor device signifies a binding event between an analyte present in the sample and the functionalized nanotubes of the working electrode, wherein the analyte is a volatile organic compound.

23. The method of claim 22, wherein the volatile organic compound is, or comprises, a biomarker selected from methyl nicotinate, methyl phenylacetate, methyl p-anisate, o-phenylanisole, or combinations thereof.

24.-25. (canceled)

26. A method of making the working electrode of claim 1, comprising:
performing a first anodization of a metal substrate to obtain the metal oxide-based nanotubes;
performing a second anodization of the metal oxide-based nanotubes to increase the length of the metal oxide-based nanotubes; and
depositing the metal ion species and/or the electroactive polymer component on the metal oxide-based nanotubes to provide the plurality of functionalized nanotubes.
